(19) United States
(12) Patent Application Publication
(10) Pub. No.: US 2010/0016638 A1
(43) Pub. Date: Jan. 21, 2010

(54) Method for preparation of o-desmethylvenlafaxine using polythiolates

(76) Inventors: Zdenko Hamersak, Zagreb (HR); Amir Avdagic, Zagreb (HR)

Correspondence Address: KENYON & KENYON LLP, ONE BROADWAY, NEW YORK, NY, 10004, US

(21) Appl. No.: 12/460,667

(22) Filed: Jul. 21, 2009

Related U.S. Application Data
(60) Provisional application No. 61/082,265, filed on Jul. 21, 2008.

(51) Int. Cl. C07C 211/27

(52) U.S. Cl. 564/366

(57) ABSTRACT

This invention relates to methods of making O-desmethylvenlafaxine from venlafaxine. In the methods venlafaxine is contacted with a polythiolate. The polythiolate may be prepared from the corresponding polythiol in the presence of base. The present methods provide the O-desmethylvenlafaxine in good yields and high purity and are suitable for use on an industrial scale.

### CROSS REFERENCE TO RELATED APPLICATIONS

The present invention claims the benefit of the following U.S. Provisional Patent Application No. 61/082,265, filed Jul. 21, 2008. The contents of this application are incorporated herein by reference.

### FIELD OF THE INVENTION

The present invention relates to methods of synthesizing O-desmethylvenlafaxine from venlafaxine using an organic polythiolate, such as an organic dithiolate.

### BACKGROUND OF THE INVENTION

Desmethylvenlafaxine succinate monohydrate, chemically known as 2-(1-hydroxycyclohexy)-2-(4-hydroxyphenyl)ethyl]dimethylammonium 3-carboxypropanoate monohydrate, is the succinate salt of a major active metabolite of venlafaxine hydrochloride. Desvenlafaxine is also known as O-desmethylvenlafaxine (ODV). ODV succinate is a serotonin-norepinephrine reuptake inhibitor (SNRI) and is approved for the treatment of adult patients with major depressive disorder (MDD). It has the following structure:

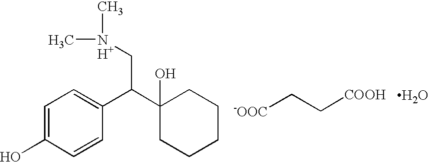

Several methods of making desmethylvenlafaxine and its salts are known. A method of making desmethylvenlafaxine and its fumarate salt was first described in U.S. Pat. No. 4,535,186. WO02/64543 described desmethylvenlafaxine succinate monohydrate and a process for its preparation. Various reagents such as lithium diphenylphosphide (WO 00/59851), L-selectride (U.S. Pat. No. 7,026,508), sodium sulfide (WO 07/071404), dodecane thiolate (U.S. Pat. No. 6,689,912), sodium ethanethiolate and thiophenol (US20080015259) have been reported for demethylation of the methoxy group of venlafaxine to give O-desmethylvenlafaxine. Other synthetic methods are described in U.S. Pat. Nos. 5,043,466; 7,141,697 and in U.S. publication Nos. 2007/0015828 and 2007/0149813. The contents of each of these references are incorporated herein by reference in their entirety.

### SUMMARY OF THE INVENTION

In one embodiment, the present invention provides methods for synthesizing O-desmethylvenlafaxine (ODV) from venlafaxine. The methods include contacting venlafaxine with an organic polythiolate under conditions suitable to provide O-desmethylvenlafaxine. The organic polythiolate may be generated from the corresponding polythiol in the presence of base. Venlafaxine may then added to the reaction mixture or the thiolate added to the venlafaxine to form ODV. Optionally, the methods further include converting the ODV obtained using such methods to a pharmaceutically acceptable salt by contacting ODV with an appropriate acid.

In specific embodiments, the present invention provides a process comprising contacting venlafaxine with 1,5-pentanedithiolate, 1,6-hexanedithiolate, 1,9-nonanedithiolate or 1,3-benzenedithiolate in a solvent to provide O-desmethylvenlafaxine

### DETAILED DESCRIPTION OF THE INVENTION

As used herein, the term “essentially pure” refers to having less than 5% by HPLC, preferably less than 2% by HPLC, more preferably less than 1% by HPLC of total impurities present.

As used herein, the term “base” refers to a chemical compound that substantially deprotonates another compound when reacted with it.

As used herein, the term “substituted” refers to an organic group (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group may be substituted with one or more substituents, unless otherwise specified.

In some embodiments, a substituted group is substituted with 1, 2, 3, 4, 5, or 6 substituents. Examples of substituent groups include: halogens (i.e., F, Cl, Br, and I); hydroxyls; alkoxy, alkenoxy, aryloxy, aralkyloxy, heterocyclyloxy, and heterocyclylalkoxy groups; carbonyls(oxo); carboxyls; esters; urethanes; oximes; hydroxylamines; alkoxyamines; aralkoxyamines; thiols; sulfides; sulfoxides; sulfones; sulfonyls; sulfonamides; amines; N-oxides; hydrazines; hydrazides; hydrazones; azides; amides; ureas; amidines; guanidines; enamines; imides; isocyanates; isothiocyanates; cyanates; thiocyanates; imines; nitro groups; nitriles (i.e., CN); and the like.

The following terms are used herein as defined below.

Alkyl groups include straight chain and branched chain alkyl groups having from 2 to 20 carbon atoms, and typically from 2 to 12 carbons or, in some embodiments, from 2 to 10, 2 to 8, or 2 to 6 carbon atoms. Examples of straight chain alkyl groups include but are not limited to groups such as methyl, ethyl, n-propyl, n-butyl, n-pentyl, n-hexyl, n-heptyl, n-octyl, n-nonyl and n-decyl groups. Examples of branched alkyl groups include, but are not limited to, isopropyl, iso-butyl, sec-butyl, tert-butyl, neopentyl, isopentyl, and 2,2-dimethylpropyl groups.

Cycloalkyl groups include mono-, bi- or tricyclic alkyl groups having from 3 to 14 carbon atoms in the ring(s), or, in some embodiments, 3 to 12, 3 to 10, 3 to 8, or 3, 4, 5, or 6 carbon atoms. Exemplary monocyclic cycloalkyl groups include, but not limited to, cyclopropyl, cyclobutyl, cyclopentyl, cyclohexyl, cycloheptyl, and cyclooctyl groups. In some embodiments, the cycloalkyl group has 3 to 8 ring members, whereas in other embodiments the number of ring carbon atoms range from 3 to 5, 3 to 6, or 3 to 7. Bi- and tricyclic ring systems include both bridged cycloalkyl groups as described below, and fused rings, such as, but not limited to, decalinyl, and the like. Alkyl groups including straight-chain, branched and cycloalkyl groups can further be substituted one or more times with, non-hydrogen groups.

Aryl groups are cyclic aromatic hydrocarbons that do not contain heteroatoms and have from 6 to 14 carbons. Aryl groups herein include monocyclic, bicyclic and tricyclic ring systems. Thus, aryl groups include, but are not limited to, phenyl, biphenyl, azulenyl, heptalenyl, fluorenyl, phenanthrenyl, anthracenyl, indenyl, indanyl, pentalenyl, and naphthyl groups. In some embodiments, aryl groups contain 6-12 carbons, and in others from 6-10 carbon atoms in the ring portions of the groups. In some embodiments, the aryl groups are phenyl or naphthyl. The phrase “aryl groups” includes groups containing fused rings, such as fused aromatic-aliphatic ring systems such as for e.g., indanyl, tetrahydronaphthyl, and the like. Aryl groups can further be substituted one or more times with, non-hydrogen groups.

Aralkyl groups are alkyl groups as defined above in which a hydrogen or carbon bond of an alkyl group is replaced with a bond to an aryl group as defined above. In some embodiments, aralkyl groups contain 7 to 14 carbon atoms, 7 to 12 carbon atoms, or 7 to 10 carbon atoms. Aralkyl groups can further be substituted one or more times with, non-hydrogen groups. Substituted aralkyl groups may be substituted at the alkyl, the aryl or both the alkyl and aryl portions of the group. Representative aralkyl groups include but are not limited to benzyl and phenethyl groups and fused (cycloalkylaryl)alkyl groups such as 4-indanylethyl.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” “about” and the like include the number recited and refer to ranges which can be subsequently broken down into subranges as discussed above. Finally, as will be understood by one skilled in the art, a range includes each individual member. Thus, for example, a group having 1-3 atoms refers to groups having 1, 2, or 3 atoms. Similarly, a group having 1-5 atoms refers to groups having 1, 2, 3, 4, or 5 atoms, and so forth.

Those of skill in the art will appreciate that compounds of the invention may exhibit the phenomena of tautomerism, conformational isomerism, geometric isomerism and/or stereoisomerism. As the formula drawings within the specification and claims can represent only one of the possible tautomeric, conformational isomeric, stereoisomeric or geometric isomeric forms, it should be understood that the invention encompasses any tautomeric, conformational isomeric, stereoisomeric and/or geometric isomeric forms of the compounds having one or more of the utilities described herein, as well as mixtures of these various different forms.

Stereoisomers of compounds (also known as optical isomers) include all chiral, diastereomeric, and racemic forms of a structure, unless the specific stereochemistry is expressly indicated. Thus, compounds used in the present invention include enriched or resolved optical isomers at any or all asymmetric atoms as are apparent from the depictions. Both racemic and diastereomeric mixtures, as well as the individual optical isomers can be isolated or synthesized so as to be substantially free of their enantiomeric or diastereomeric partners, and these stereoisomers are all within the scope of the invention. For e.g., pharmaceutically acceptable salts of O-desmethylvenlafaxine can exist as enantiomers and this invention includes racemic mixtures as well as stereoisomerically pure forms of the same.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

By way of illustrating, the process provided by the present invention, of synthesizing O-desmethylvenlafaxine (ODV) from venlafaxine can be schematically represented as:

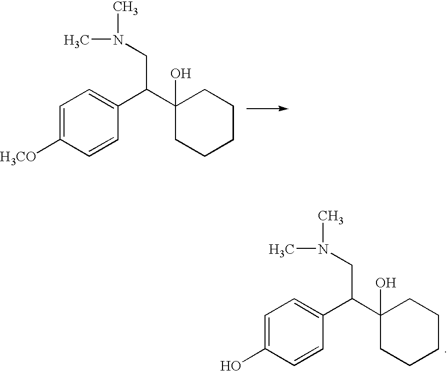

The methods include contacting venlafaxine with an organic polythiolate under conditions suitable to provide O-desmethylvenlafaxine. The starting material, venlafaxine, can be prepared by methods known in the art, such as, e.g., those described in U.S. Pat. Nos. 4,535,186 and 5,043,466. The organic polythiolate may be generated from the corresponding polythiol in the presence of base. Venlafaxine may then be added to the reaction mixture or the thiolate may be added to the venlafaxine to form ODV. The demethylation reaction may be conducted at elevated temperature. The reaction can be monitored for disappearance of the starting material i.e. venlafaxine by various analytical methods such as, e.g., TLC or HPLC. The reaction mixture may be worked up by standard techniques known to those skilled in the art such as, e.g., extraction of basic and acidic solutions of the reaction mixture with suitable solvents, to provide ODV in substantially pure form. Optionally, the methods further include converting the ODV obtained using such methods to a pharmaceutically acceptable salt by contacting ODV with an appropriate acid. Preferably, the process of present invention provides ODV with high purity and in high yield as compared to the previous processes (see comparative example 14). Typical yields of ODV using the present methods are greater than about 40% and mostly at least about 70%; and in some embodiments, are at least about 80% by weight. The corresponding pharmaceutically acceptable salt such as, for e.g., the succinate salt is also obtained with improved yields.

A wide range of organic polythiolates are suitable for use in the present methods. Organic polythiolates as defined herein are carbon-containing compounds having at least two thiol groups (—SH), one or more of which is deprotonated, i.e., a thiolate group (—S⁻). It will be appreciated by those skilled in the art that under the reaction conditions described herein, organic polythiolates exist as a mixture of interconverting species in which most, but not necessarily all of the thiol groups are ionized to thiolate groups. Nonetheless, the species will be collectively referred to herein as polythiolates. In some embodiments, the organic polythiolates have two to six thiolate groups such as, e.g., dithiolates, trithiolates, tetrathiolates, pentathiolates, and hexathiolates. In some embodiments, the organic polythiolate has from 2 to 20 carbon atoms and can be, e.g., an alkyl, cycloalkyl, aryl or aralkyl polythiolate. In some embodiments of the present methods, the polythiolate can be an alkyl dithiolate, cycloalkyl dithiolate, aryl dithiolate, or aralkyl dithiolate. Preferably, the dithiolate is selected from the group consisting of: pentanedithiolate, hexanedithiolate, nonanedithiolate and benzenedithiolate. More preferably, the dithiolate is selected from the group consisting of: 1,6-hexanedithiolate, 1,5-pentanedithiolate, 1,9-nonanedithiolate and 1,3-benzenedithiolate. Most preferably, the dithiolate is 1,6-hexanedithiolate or 1,3-benzenedithiolate. Thus, organic polythiolates used herein may have 2 or more carbon atoms linking but separating any two thiolate groups.

The polythiolates of the present invention can be generated independently or in-situ. In one aspect of the invention, the polythiolate anion is produced in-situ by contacting a polythiol with a base.

In some embodiments, the base that may be used to generate a polythiolate anion can be selected from the group consisting of: hydroxide, alkoxide and hydride of an alkali metal or an alkaline earth metal. For example, the base can be sodium, potassium or lithium hydroxide, sodium, potassium or lithium hydride, or sodium or potassium alkoxide such as, but not limited to, sodium or potassium t-butoxide, propoxide, i-propoxide, ethoxide, methoxide, and the like. Preferably, the base is selected from the group consisting of: sodium methoxide, potassium tert-butoxide, sodium hydroxide, lithium hydroxide and sodium hydride. More preferably, the base is sodium methoxide or potassium tert-butoxide. The amount of base varies with the amount of polythiol and the number of thiol groups, but is typically in molar excess relative to the molar amount of polythiol but substoichiometric with respect to the molar amount of thiol groups. For example, in some embodiments, 1.5 to 1.9 equivalents of base may be used per equivalent of dithiol. In other embodiments 1.5 to 2.5 equivalents of base may be used per equivalent of trithiols. The base may be added to a solution of polythiol in a suitable solvent to provide, e.g., an alcoholic or aqueous solution of the base. Suitable alcohols comprise a straight or branched chain alkyl group of 1 to 6 carbon atoms. Preferably, the suitable alcohol is selected from the group consisting of: methanol, ethanol, iso-propanol, butanol, pentanol, hexanol and mixtures thereof.

In one embodiment, the process of contacting venlafaxine with a polythiolate is conducted in presence of a suitable solvent or solvents. Suitable solvents for use in the present methods include both protic and aprotic solvents. Suitable solvents are selected from the group comprising of: chlorinated aliphatic solvents such as chloroform, 1,1-dichloroethane, etc.; hydrocarbon solvents such as toluene, xylene etc.; amide solvents such as dimethylformamide, dimethylacetamide, etc.; sulfoxides such as dimethylsulfoxide, etc.; and ether solvents such as polyethylene glycol, N-methyl pyrrolidinone (NMP) etc. Preferably, a suitable solvent would be one in which both venlafaxine and polythiolate are soluble and which has a boiling point greater than about 100° C. In some embodiments lower boiling solvents such as chloroform and dichloroethane can be employed. Suitable solvents include but are not limited to diethylene glycol, triethylene glycol, polyethylene glycol 200-600, N-methyl pyrrolidinone (NMP), dimethylformamide, dimethylacetamide, dimethylsulfoxide or mixtures thereof. Preferably, the solvent is selected from polyethylene glycol 400 or N-methyl pyrrolidinone (NMP). More preferably, the solvent is N-methyl pyrrolidinone (NMP). The amount of solvents varies with the starting materials.

The present methods may be carried out a range of concentrations and stoichiometries of the reactants. For example, the reaction may be carried out at concentrations of venlafaxine ranging from about 0.1 M to about 3 M, from about 0.2 M to about 3 M, from about 0.5 M to about 3 M or even higher.

In some embodiments, the molar ratio of the starting materials ranges from about 0.1 to about 4 mol of polythiol per mol of venlafaxine.

In other embodiments, the molar ratio of the starting materials ranges from about 0.5 to about 3.5 moles of polythiol per mol of venlafaxine.

In illustrative embodiments, the molar ratio of the starting materials varies from about 0.75 to about 3.5, or from about 0.75 to about 1.5 or about 2 moles moles of polythiol per mole of venlafaxine.

Typically the present methods are carried out at elevated temperatures, e.g., from about 100° C. to about 220° C. In one embodiment the reaction is performed at about 130° C. to about 190° C. Preferably, the process of contacting venlafaxine with a polythiolate is conducted at a temperature of from about 140° C. to about 150° C.

The reaction is typically conducted for a time period sufficient for the reaction to go to completion. The reaction can be monitored for disappearance of the starting material i.e. venlafaxine by various analytical methods such as, e.g., TLC or HPLC. Preferably, the process of contacting venlafaxine with a polytiolate is conducted for about 5 hr to about 48 hr. More preferably, the time period is from about 10, 15, or 20 hr to about 24 or 36 hr.

The present methods provide substantially pure ODV, an important consideration on an industrial scale. Purity of the ODV may conveniently be assessed by HPLC. In some embodiments, ODV obtained by the present methods exhibits purity of greater than about 90% area by HPLC. Preferably, the purity of the ODV is at least about 95% area by HPLC. More preferably, the purity of the ODV is at least about 98%. Most preferably, the purity of the ODV is at least about 99.5% area by HPLC.

The O-desmethylvenlafaxine obtained by the present methods may be converted to a pharmaceutically acceptable salt by contacting the ODV to an acid. Pharmaceutically acceptable salts can be formed with an acid selected from the group consisting of: hydrochloric acid, hydroboric acid, nitric acid, sulfuric acid, phosphoric acid, succinic acid, formic acid, acetic acid, trifluoroacetic acid, fumaric acid, oxalic acid, tartaric acid, lactic acid, maleic acid, citric acid, malic acid, methanesulfonic acid, benzenesulfonic acid, p-toluenesulfonic acid, aspartic acid and glutamic acid. Preferably, the pharmaceutically acceptable salt is an acetate, benzenesulfonate, benzoate, camphorsulphonate, citrate, ethanesulfonate, fumarate, hydrobromate, hydrochlorate, lactate, maleate, malate, mandelate, methanesulphonate, nitrate, pamoate, pantothenate, phosphorate, succinate, tartarate or p-toluenesulfonate, tartrate, alkylsulfonate, arysulfonate, sulfate, phosphate, hydrochloride, hydrobromide, sulfamate, maleate, lactate, malic, gluconate, or an ascorbate. More preferably, the salt is a succinate salt.

In one embodiment, the present invention provides a process comprising contacting venlafaxine with 1,5-pentanedithiolate, 1,6-hexanedithiolate, 1,9-nonanedithiolate or 1,3-benzenedithiolate in a solvent to provide O-desmethylvenlafaxine.

Preferably, the solvent for the dissolution includes but is not limited to diethylene glycol, triethylene glycol, polyethylene glycol 200-600, N-methyl pyrrolidinone (NMP), dimethylformamide, dimethylacetamide, dimethylsulfoxide or mixtures thereof More preferably the solvent is selected from polyethylene glycol 400 or N-methyl pyrrolidinone (NMP). Most preferably, the solvent is N- methyl pyrrolidinone (NMP).

The dithiolate can be formed in-situ by contacting the dithiol with a base or can be employed, as such, into the reaction mixture. Preferably, the base is selected from the group consisting of: sodium methoxide, potassium tert-butoxide, sodium hydroxide, lithium hydroxide and sodium hydride. More preferably, the base is lithium hydroxide or sodium hydride

In one specific embodiment, the present invention provides a process for the preparation of ODV succinate salt comprising dissolving dithiol in a solvent, to which an aqueous or an alcoholic solution of a base is added. After stirring the obtained reaction mixture for a time suitable to dissolve the reagents and to allow dithiolate formation, venlafaxine is added to the reaction mixture. The alcohol or water is distilled off and the reaction mixture is stirred at a temperature of about 130° C. to about 190° C. for about 3 to about 48 hrs. The reaction mixture is cooled and worked-up according to standard techniques known in the art. O-desmethylvenlafaxine is obtained with high yield and high purity. ODV is subsequently suspended in a suitable solvent or a mixture thereof to which succinic acid is added. The suspension is heated to reflux until a clear solution is obtained. It is then filtered, and stirred at lower temperatures to give the succinate salt of ODV.

Preferably, the dithiol is selected from the group consisting of: pentanedithiol, hexanedithiol, nonanedithiol and benzenedithiol. More preferably, the dithiol is selected from the group consisting of: 1,6-hexanedithiol, 1,5-pentanedithiol, 1,9-nonanedithiol and 1,3-benzenedithiol. Most preferably, the dithiol is 1,6-hexanedithiol.

Preferably, the solvent for the dissolution includes but are not limited to diethylene glycol, triethylene glycol, polyethylene glycol 200-600, N-methyl pyrrolidinone (N), dimethylformamide, dimethylacetamide, dimethylsulfoxide or mixtures thereof. More preferably the solvent is selected from polyethylene glycol 400 or N-methyl pyrrolidinone (NMP). Most preferably, the solvent is N-methyl pyrrolidinone (NMP).

Preferably, the base is selected from the group consisting of: sodium methoxide, potassium tert-butoxide, sodium hydroxide, lithium hydroxide and sodium hydride. More preferably, the base is sodium methoxide or potassium tert-butoxide.

Preferably, the solvent for the suspension of ODV is acetone and water or a mixture thereof.

Preferably, the reaction mixture is stirred at a temperature of about 135° C. to about 150° C. More preferably, at a temperature of about 140° C.

The examples herein are provided to illustrate advantages of the present invention and to further assist a person of ordinary skill in the art with preparing or using the compounds of the invention or salts, pharmaceutical compositions, derivatives, metabolites, prodrugs, racemic mixtures or tautomeric forms thereof. The examples herein are also presented in order to more fully illustrate the preferred aspects of the invention. The examples should in no way be construed as limiting the scope of the invention, as defined by the appended claims. The examples can include or incorporate any of the variations, aspects or aspects of the invention described above. The variations, aspects or aspects described above may also further each include or incorporate the variations of any or all other variations, aspects or aspects of the invention.

The present invention, thus generally described, will be understood more readily by reference to the following examples, which are provided by way of illustration and are not intended to be limiting of the present invention.

### Examples

The following abbreviations are used throughout the present disclosure with respect to chemical terminology:
DCM: Dichloromethane
DEG: Diethyleneglycol
DIPE: Diisopropyl ether
DMA: N,N-dimethylacetamide
DMF: N,N-Dimethylformamide
DMSO: Dimethylsulfoxide
EtOAc: Ethyl acetate
H₃PO₄: Phosphoric acid
MeOH: Methanol
mL: Milliliter(s)
mmol: Millimole(s)
nm: nanometer
NMP: N-methyl pyrrolidinone
NMR: Nuclear magnetic resonance
ODV: O-Desmethylvenlafaxine
PEG-400 Polyethylene glycol 400
TEG Triethylene glycol

HPLC conditions: Agilent 1090 instrument, Nucleosil 100-5 RP18 column, gradient 10% MeOH+90% of 0.5% H₃PO₄ to 100% MeOH in 20 min., detection at 229 nm.

### Example 1

To a solution of 1,6-hexanedithiol (2.5 g; 16.6 mmol) in 6 ml of NMP, 5.0 ml (27.7 mmol) of a 30% methanolic solution of sodium methoxide was added. After stirring the solution for 10 min, venlafaxine (4.5 g; 16.2 mmol) was added. Methanol was distilled off and the reaction mixture was stirred at 140° C. for 24 hours. The temperature was then lowered to 70° C. and 70 ml of 2% NaOH was added. The resulting basic aqueous solution was washed with diisopropylether and acidified with conc. HCl to pH 1-2. The acidic solution was washed again with diisopropylether. Residual solvent was removed under vacuum; the aqueous solution was filtered and the pH was adjusted to 9.5-10 with aqueous ammonia. Precipitated product was collected on a filter, washed with water and dried. Yield: 3.44 g (80%); HPLC: 99%.

### Example 2

To a solution of 1,6-hexanedithiol (2.5 g; 16.6 mmol) in 5 ml of PEG-400, 5.5 ml (30.4 mmol) of 30% methanolic solution of sodium methoxide was added. After stirring the solution for 10 min, venlafaxine (5.0 g; 18.0 mmol) was added. Methanol was distilled off and the reaction mixture was stirred at 145° C. for 23 hours. The temperature was then was lowered to 70° C. and 70 ml of 2% NaOH was added. The resulting basic aqueous solution was washed with diisopropylether and acidified with conc. HCl to pH 1-2. The acidic solution was washed again with diisopropylether. Residual solvent was removed by vacuum; aqueous solution was filtered and pH was adjusted to 9.5-10 with aqueous ammonia. Precipitated product was collected on a filter, washed with water and dried. Yield: 3.87 g, 81.5%, HPLC: 99.5%.

### Example 3

To a solution of 1,6-hexanedithiol (2.5 g; 16.6 mmol) in 5 ml of PEG-400, 5.0 ml (27.7 mmol) of 30% methanolic solution of sodium methoxide was added. After stirring the solution for 10 min, venlafaxine (4.5 g; 16.2 mmol) was added. Methanol was distilled off and the reaction mixture was stirred at 130° C. for 48 hours. The temperature was then lowered to 70° C. and 70 ml of 2% NaOH was added. The resulting basic aqueous solution was washed with diisopropylether and acidified with conc. HCl to pH 1-2. The acidic solution was washed again with diisopropylether. Residual solvent was removed by vacuum; aqueous solution was filtered and pH was adjusted to 9.5-10 with aqueous ammonia. Precipitated product was collected on filter, washed with water and dried. Yield: 3.25 g (75%); HPLC: 98%.

### Example 4

To a solution of 1,6-hexanedithiol (2.5 g; 16.6 mmol) in 7 ml of NMP, 5.0 (27.7 mmol) ml of 30% methanolic solution of sodium methoxide was added. After stirring the solution for 10 min, venlafaxine (6.0 g; 21.6 mmol) was added. Methanol was distilled off and the reaction mixture was stirred at 190° C. for 5 hours. The temperature was then lowered to 70° C. and 90 ml of 2% NaOH was added. The resulting basic aqueous solution was washed with diisopropylether and acidified with conc. HCl to pH 1-2. The acidic solution was washed again with diisopropylether. Residual solvent was removed by vacuum; aqueous solution was filtered and pH was adjusted to 9.5-10 with aqueous ammonia. Precipitated product was collected on filter, washed with water and dried. Yield: 4.80 g (84%); HPLC: 91%.

### Example 5

To a solution of 1,9-nonanedithiol (3.23 g; 16.6 mmol) in 5 ml of PEG-400, 5.0 ml (27.7 mmol) of 30% methanolic solution of sodium methoxide was added. After stirring the solution for 10 min venlafaxine (4.5 g; 16.2 mmol) was added. Methanol was distilled off and the reaction mixture was stirred at 140° C. for 24 hours. The temperature was then lowered to 70° C. and 70 ml of 2% NaOH was added. The resulting basic aqueous solution was washed with diisopropylether and acidified with conc. HCl to pH 1-2. The acidic solution was washed again with diisopropylether. Residual solvent was removed by vacuum; aqueous solution was filtered and pH was adjusted to 9.5-10 with aqueous ammonia. Precipitated product was collected on filter, washed with water and dried. Yield: 3.57 g (83%); HPLC: 93%.

### Example 6

To a solution of 1,5-pentanedithiol (2.25 g; 16.6 mmol) in 5 ml of PEG-400, 5.0 ml (27.7 mmol) of 30% methanolic solution of sodium methoxide was added. After stirring the solution for 10 min, venlafaxine (4.5 g; 16.2 mmol) was added. Methanol was distilled off and the reaction mixture was stirred at 140° C. for 24 hours. The temperature was then lowered to 70° C. and 70 ml of 2% NaOH was added. The resulting basic aqueous solution was washed with diisopropylether and acidified with conc. HCl to pH 1-2. The acidic solution was washed again with diisopropylether. Residual solvent was removed by vacuum; aqueous solution was filtered and pH was adjusted to 9.5-10 with aqueous ammonia. Precipitated product was collected on filter, washed with water and dried. Yield: 3.42 g (79%); HPLC: 98%.

### Example 7

To a solution of 1,3-benzenedithiol (1.77 g; 12.6 mmol) in 5 ml of PEG-400, 3.7 ml (20.5 mmol)of 30% methanolic solution of sodium methoxide was added. After stirring the solution for 10 min, venlafaxine (3.4 g; 12.6 mmol) was added. Methanol was distilled off and the reaction mixture was stirred at 145° C. for 24 hours. The temperature was then lowered to 70° C. and 70 ml of 2% NaOH was added. The resulting basic aqueous solution was washed with diisopropylether and acidified with conc. HCl to pH 1-2. The acidic solution was washed again with diisopropylether. Residual solvent was removed by vacuum; aqueous solution was filtered and pH was adjusted to 9.5-10 with aqueous ammonia. Precipitated product was collected on filter, washed with water and dried. Yield: 2.51 g (77%); HPLC: 97%.

### Example 8

To a solution of 1,6-hexanedithiol (2.5 g; 16.6 mmol) in 10 ml of PEG-400 2.1 g (30.4 mmol) of 60% NaH dispersion in mineral oil was gradually added. After evolution of hydrogen has cased, venlafaxine (5.0 g; 18.0 mmol) was added. The reaction mixture was stirred at 145° C. for 20 hours. The temperature was then lowered to 70° C. and 70 ml of 2% NaOH was added. The resulting basic aqueous solution was washed with DCM (dichloromethane) and acidified with conc. HCl to pH 2. The acidic solution was washed again with DCM. Residual solvent was removed by vacuum; aqueous solution was filtered and pH was adjusted to 9.5-10 with aqueous ammonia. Precipitated product was collected on filter, washed with water and dried. Yield: 3.33 g (72% %); HPLC: 99.0%.

### Example 9

To a solution of 1,6-hexanedithiol (1.5 g; 10.0 mmol) in 5 ml of PEG-400 0.5 g (12.5 mmol) of NaOH in 0.8 ml of water was added. After stirring the solution for 10 min, venlafaxine (1.5 g; 5.4 mmol) was added. Water was distilled off and the reaction mixture was stirred at 145° C. for 24 hours. The temperature was then lowered to 70° C. and 40 ml of 2% NaOH was added. The resulting basic aqueous solution was washed with DCM and acidified with conc. HCl to pH 1-2. The acidic solution was washed again with DCM. Residual solvent was removed by vacuum; aqueous solution was filtered and pH was adjusted to 9.5-10 with aqueous ammonia. Precipitated product was collected on filter, washed with water and dried. Yield: 850 mg (60%); HPLC: 96%.

### Example 10

To a solution of 1,6-hexanedithiol (1.0 g; 6.6 mmol) in 5 ml of PEG-400 5.5 g (5.4 mmol) potassium t-butoxide was added. After stirring the solution for 10 min, venlafaxine (0.8 g; 2.9 mmol) was added. The reaction mixture was stirred at 145° C. for 20 hours. The temperature was then lowered to 70° C. and 30 ml of 2% NaOH was added. The resulting basic aqueous solution was washed with DCM and acidified with conc. HCl to pH 1-2. The acidic solution was washed again with DCM. ;Residual solvent was removed by vacuum; aqueous solution was filtered and pH was adjusted to 9.5-10 with aqueous ammonia. Precipitated product was collected on filter, washed with water and dried. Yield: 320 mg (42%); HPLC: 95%.

### Example 11

To a solution of 1,6-hexanedithiol (1.5 g; 10.0 mmol) in 5 ml of PEG-400 0.3 g (12.5 mmol) of LiOH in 0.7 ml of water was added. After stirring the solution for 10 min. venlafaxine (0.8 g; 2.9 mmol) was added. Water was distilled off and the reaction mixture was stirred at 145° C. for 20 hours. The temperature was then lowered to 70° C. and 30 ml of 2% NaOH was added. The resulting basic aqueous solution was washed with DCM and acidified with conc. HCl to pH 1-2. The acidic solution was washed again with DCM. Residual solvent was removed by vacuum; aqueous solution was filtered and pH was adjusted to 9.5-10 with aqueous ammonia. Precipitated product was collected on filter, washed with water and dried. Yield: 340 mg (45%); HPLC: 96%.

### Example 12

### Preparation of Succinate Salt of ODV

To a stirred suspension of 3.42 g (13.0 mmol) of O-desmethylvenlafaxine in 33 ml of acetone and 10 ml of water 1.58 g (13.4 mmol) of succinic acid was added. The suspension was heated to reflux until a clear solution was obtained, filtered and allowed to cool down to 30° C. The suspension was stirred at 30° C. for another 3 hours and then at 0° C. for 3 hours. Product was filtered, washed with cold 75% acetone and dried. Yield: 3.82 g (77%).

### Example 13

To a solution of 1,6-hexanedithiol (2.5 g; 16.6 mmol) in 5 ml of PEG-400, 5.5 ml (30.4 mmol) of 30% methanolic solution of sodium methoxide was added. After stirring the solution for 10 min, venlafaxine (5.0 g; 18.0 mmol) was added. Methanol was distilled off and the reaction mixture was stirred at 145° C. for 20 hours. The temperature was then lowered to 70° C. and 70 ml of 2% NaOH was added. The resulting basic aqueous solution was washed with DCM and acidified with conc. HCl to pH 2. The acidic solution was washed again with DCM. Residual solvent was removed by vacuum; aqueous solution was filtered and pH was adjusted to 9.5-10 with aqueous ammonia. Precipitated product was collected on filter, washed with water and dried. Yield: 3.48 g (74% %); HPLC: 99.5%;

To a stirred suspension of 3.48 g (13.2 mmol) of O-desmethylvenlafaxine (obtained by the above process) in 33 ml of acetone and 10 ml of water 1.61 g (13.6 mmol) of succinic acid was added. The suspension was heated to reflux until clear solution was obtained, filtered and allowed to cool down to 30° C. The suspension was stirred at 30° C. for another 3 hours and then at 0° C. for 3 hours. Product was filtered, washed with cold 75% acetone and dried. Yield: 34.45 g (84%).

### Example 14

### Comparative Example

To a solution of 1-dodecylmercaptane (6.7 g; 33 mmol) in 6 ml of PEG-400, 5.5 ml (30.4 mmol) of 30% methanolic solution of sodium methoxide was added. After stirring the solution for 10 min, venlafaxine 5.0 g; 18.0 mmol was added. Methanol was distilled off and the reaction mixture was stirred at 190° C. for 4 hours. The reaction was monitored by HPLC every 30 min for disappearance of venlafaxine; at 4 hours the reaction was 90% complete. (More extended heating produced greater amounts of impurities.) The temperature was then lowered to 70° C. and 70 ml of 2% NaOH was added. The resulting basic aqueous solution was washed with DCM and acidified with conc. HCl to pH 2. The solution was washed again with DCM. Residual solvent was removed by vacuum; aqueous solution was filtered and pH was adjusted to 9.5-10 with aqueous ammonia. Precipitated product was collected on filter, washed with water and dried. Yield: 3.0 g (63%); HPLC: 98.0%.

When this demethylation reaction was run using lower molar ratios of reactants according to Example 1 of U.S. Pat. No. 6,689,912, the conversion to product stalled at 70% and did not go to completion.

To a stirred suspension of 3.00 g (11.4 mmol) of O-desmethylvenlafaxine in 30 ml of acetone and 9 ml of water, 1.4 g (11.8 mmol) of succinic acid was added. The suspension was heated to reflux until a clear solution was obtained, filtered and allowed to cool down to 30° C. The suspension was stirred at 30° C. for another 3 hours and then for 3 hours at 0° C. Product was filtered, washed with cold 75% acetone and dried. Yield: 3.42 g (75%).

What is claimed is:

1. A method comprising contacting venlafaxine with an organic polythiolate and a solvent to provide O-desmethylvenlafaxine.

2. The method of claim 1, wherein the organic polythiolate has two to six thiolate groups.

3. The method of claim 2, wherein the organic polythiolate has two thiolate groups.

4. The method of claim 3, wherein the polythiolate is selected from the group comprising of alkyl dithiolate, cycloalkyl dithiolate, aryl dithiolate, and an aralkyl dithiolate.

5. The method of claim 4, wherein the polythiolate is an alkyl dithiolate having 4 to 12 carbon atoms or an aralkyl dithiolate having 7 to 14 carbon atoms.

6. The method of claim 4, wherein the polythiolate is selected from the group consisting of 1,6-hexanedithiolate, 1,3-benzenedithiolate, 1,9-nonanedithiol and 1,5-pentanedithiol.

7. The method of claim 4, wherein the polythiolate is 1,6-hexanedithiol.

8. The method of claim 1, wherein the solvent is selected from diethylene glycol, triethylene glycol, polyethylene glycol, N-methyl pyrrolidinone, dimethylformamide, dimethylacetamide, dimethylsulfoxide or a mixture of two or more thereof.

9. The method of claim 8, wherein the solvent is polyethylene glycol 400 or N-methylpyrrolidinone.

10. The method of claim 8, wherein the solvent is N-methyl pyrrolidinone.

11. The method of claim 1, wherein the polythiolate anion is generated in-situ in presence of a base.

12. The method of claim 11, wherein the base is selected from a hydroxide, alkoxide or hydride of an alkali metal or alkaline earth metal.

13. The method of claim 12, wherein the base is selected from sodium methoxide, potassium tert-butoxide, sodium hydroxide, lithium hydroxide or sodium hydride.

14. The method of claim 12, wherein the base is sodium hydroxide, sodium methoxide or potassium tert-butoxide.

15. The method of claim 12, wherein the base is selected from, lithium hydroxide or sodium hydride.

16. The method of claim 1, wherein the reaction is performed at a temperature from about 100° C. to about 220° C.

17. The method of claim 16, wherein the reaction is performed at about 130° C. to about 190° C.

18. The method of claim 17, wherein the reaction is preformed at about 130° C. to 150° C.

19. The method of claim 18, wherein the reaction is preformed at about 140° C.-150° C.

20. The method of claim 1, further comprising contacting the O-desmethylvenlafaxine with an acid to provide a pharmaceutically acceptable salt.

21. The method of claim 20 wherein the pharmaceutically acceptable salt is an acetate, benzenesulfonate, benzoate, camphorsulphonate, citrate, ethanesulfonate, fumarate, hydrobromate, hydrochlorate, lactate, maleate, malate, mandelate, methanesulphonate, nitrate, pamoate, pantothenate, phosphorate, succinate, tartarate or p-toluenesulfonate, tartarate, alkylsulfonate, or arysulfonate, sulfate, phosphate, hydrochloride, hydrobromide, sulfamate, maleate, lactate, malic, gluconate, or an ascorbate.

22. The method of claim 20 wherein the pharmaceutically acceptable salt is a succinate salt.

23. A method comprising contacting venlafaxine with 1,6-hexanedithiol in PEG-400 in the presence of sodium methoxide to provide O-desmethylvenlafaxine.

24. The method of claim 23 wherein the reaction is performed at a temperature from about 130° C. to about 150° C.

25. The method of claim 23 further comprising contacting O-desmethylvenlafaxine free base with succinic acid to form a succinate salt.

26. A method comprising combining venlafaxine with 1,6-hexanedithiol in N-methyl pyrrolidinone in the presence of a base to provide O-desmethylvenlafaxine.

27. The method of claim 26, wherein the base is sodium hydroxide, lithium hydroxide or sodium hydride.

28. The method of claim 26, wherein the reaction is preformed at a temperature from about 130° C. to about 150° C.

29. The method of claim 26, wherein the reaction is preformed at a temperature of about 130° C.

30. The method of claim 26 further comprising contacting O-desmethylvenlafaxine free base with succinic acid to form a succinate salt.
